(19) United States
(12) Patent Application Publication
(10) Pub. No.: US 2010/0173426 A1
(43) Pub. Date: Jul. 8, 2010

(54) BIOMAKER IDENTIFYING THE REACTIVATION OF STAT3 AFTER SRC INHIBITION

(76) Inventors: Faye M. Johnson, The Woodlands, TX (US); Nicholas J. Donato, Ann Arbor, MI (US); Francis Y. Lee, Yardley, PA (US)

Correspondence Address: NIELSEN IP LAW LLC, 1177 West Loop South, Suite 1600, Houston, TX, 77027, US

(21) Appl. No.: 12/519,848

(22) PCT Filed: Dec. 18, 2007
(86) PCT No.: PCT/US2007/087982

§ 371 Date: Jan. 21, 2010

Related U.S. Application Data
(60) Provisional application No. 60/870,737, filed on Dec. 19, 2006.

(51) Int. Cl. G01N 33/574

(52) U.S. Cl. 436/501

(57) ABSTRACT

A method of identifying cancer or an associated disorder comprising identifying and quantifying STAT3 occurring in a biological sample taken from a subject after administering a SFK inhibitor to said subject.

### CROSS-REFERENCE TO RELATED APPLICATIONS

This application claims priority to U.S. Pat. App. Ser. No. 60/870,737 filed Dec. 19, 2006. Applications are incorporated by reference herein in their entirety.

### FIELD OF THE INVENTION

Methods of detecting STAT3 reactivation after the administration of a Src inhibitor to a subject in need thereof is disclosed herein.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO SEQUENCE LISTING

None.

### BACKGROUND OF THE INVENTION

Cancer is one of the principal causes of death in developed countries. Cancer may affect people at all ages, but risk tends to increase with age. The disease state is typically characterized as uncontrolled cell division coupled with the ability of these cells to invade other tissues, either by direct growth into adjacent tissue through invasion or by spreading into distant sites by a process called metastasis.

A definitive diagnosis of cancer usually requires histologic examination of tissue by a pathologist. This tissue is obtained by biopsy or surgery. Most cancers can be treated and some cured, depending on the specific type, location, and stage. Once diagnosed, cancer is usually treated with a combination of surgery, chemotherapy and radiotherapy. As research develops, treatments are becoming more specific for the type of cancer pathology. Drugs that target specific cancers already exist for several cancers. Generally, if untreated, cancers may eventually cause illness and death, though this is not always the case.

The unregulated growth that characterizes cancer is often caused by mutations to genes that encode for proteins controlling cell division. Many mutation events may be required to transform a normal cell into a malignant cell. The protein agents governing cell proliferation include a variety of cell signaling molecules such as the kinase family of enzymes responsible for carrying out phosphorylation of their target structures.

The Src family of kinases have been implicated in cancer, immune system dysfunction and bone diseases such as osteoporosis. Thomas et al., Annu. Rev. Cell Dev. Biol. (1997) 13, 513; Lawrence et al., Pharmacol. Ther. (1998) 77, 81; Tatosyan et al., Biochemistry (Moscow) (2000) 65, 49; Boschelli et al., Drugs of the Future (2000), 25(7), 717.

SFKs and certain growth factor receptors are overexpressed in various cancers. Halpern M. S., England J. M., Kopen G. C., Christou A. A., Taylor R. L. Jr., Endogenous c-src as a Determinant of the Tumorigenicity of src Oncogenes, Proc Natl Acad Sci USA. 1996 93(2): 824-827. Haura, E. B., Zheng, Z., Song, L., Cantor, A., Bepler, G., Activated Epidermal Growth Factor Receptor-Stat-3 Signaling Promotes Tumor Survival In Vivo in Non-Small Cell Lung Cancer, Clin. Cancer Res. 2005, 11(23): 8288-8294. Likewise, the activation paradigm and role of STATs (signal transducers and activators of transcription proteins) in certain cancers has been reported. See Yu, H., Jove, R., The Stats of Cancer—New Molecular Targets Come of Age, Nature Rev. 2004, 4: 97-106.

In addition, at least one member of the Src family of kinases (SFKs), c-Src, reportedly induces STATs involved in the tumorigenesis process. Xi, S., Zhang, Q., Dyer, K. F., Lerner, E. C., Smithgall, T. E. Gooding, W. E., Kamens, J., Grandis, J. R., Src Kinases Mediate STAT Growth Pathways in Squamous Cell Carcinoma of the Head and Neck, J. Biol. Chem. 2003, 278(34): 31574-31583. STAT3 is a member of the signal transducer and activator of transcription protein family that regulates many aspects of cell growth, survival and differentiation. Constitutive STAT3 has been associated with various human cancers and commonly suggests poor prognosis as it has anti-apoptotic as well as proliferative effects. Yu, H. and Jove, R. The STATs of Cancer—New Molecular Targets Come of Age, Nat Rev Cancer, 4: 97-105, 2004.

A need exists, therefore, for a method of detecting cancer by identifying STAT3 reactivation after Src inhibition.

### SUMMARY OF INVENTION

Methods of detecting STAT3 reactivation after Src inhibition are provided herein. The methods of identifying the reactivation comprises the steps of identifying and quantifying the amount of STAT3 expressed after an inhibitor of Src is administered to a subject in need thereof.

### BRIEF DESCRIPTION OF THE DRAWINGS

FIG. 1A is Western blot showing Src inhibition and STAT3 inhibition and reactivation.

FIG. 1B is Western blot showing Src inhibition and STAT3 inhibition and reactivation.

### DETAILED DESCRIPTION OF THE INVENTION

Methods for detecting STAT3 reactivation after SFK inhibition are disclosed herein. The methods of identifying cancer associated disorders comprise the steps of identifying and quantifying the amount of STAT3 expressed after an inhibitor of Src.

The Src family of kinases (“SFKs”) have multiple substrates that lead to diverse biologic effects including changes in proliferation, motility, invasion, survival and angiogenesis. The role of SFKs in the initiation and/or progression of cancer has been demonstrated in colon cancer, pancretic cancer, breast cancer, non-small cell lung cancer (NSCLC), head and neck squamous cell carcinoma (HNSCC), prostate cancer, other solid tumors, several hematologic malignancies, hepatic cancer, certain B-cell leukemias and lymphomas. Talamonti et al., J. Clin. Invest., 91, 53 (1993); Lutz et al., Biochem. Biophys. Res. 243, 503 (1998); Rosen et al., J. Biol. Chem., 261, 13754 (1986); Bolen et al., Proc. Natl. Acad. Sci. USA, 84, 2251 (1987); Masaki et al., Hepatology, 27, 1257 (1998); Biscardi et al., Adv. Cancer Res., 76, 61 (1999); and Lynch et al., Leukemia, 7, 1416 (1993). The methods and compositions disclosed herein may be used in any one or more cancers or carcinoma disorders.

A tyrosine kinase is an enzyme that transfers a phosphate group from ATP to a tyrosine residue in a protein. Tyrosine kinases are a subgroup of the larger class of protein kinases. Fundamentally, a protein kinase is an enzyme that modifies a protein by chemically adding phosphate groups to a hydroxyl or phenolic functional group. Such modification often results in a functional change to the target protein or substrate by altering the enzyme structure, activity, cellular location or association with other proteins. Chemically, the kinase removes a phosphate group from ATP and covalently attaches it to one of three amino acids (serine, threonine or tyrosine) that have a free hydroxyl group. Many kinases act on both serine and threonine, and certain others, tyrosine. There are also a number of kinases that act on all three of these amino acids.

Tyrosine kinases are divided into two groups: cytoplasmic proteins and transmembrane receptor kinases. In humans, there are 32 cytoplasmic protein tyrosine kinases and 48 receptor-linked protein-tyrosine kinases.

Generally, tyrosine kinases play critical roles in signaling between cells. Basically, the activation of cell surface receptors (e.g., the epidermal growth factor (EGF) receptor) by extracellular ligands results in the activation of tyrosine kinases. Then, the tyrosine kinase generates phosphotyrosine residues in the cell. The phosphotyrosine residue acts as a “beacon” and attracts signaling proteins to the receptor via SH2 domains. Hence, one important aspect of the signaling mechanism of a tyrosine kinase is the recognition of the phosphotyrosine by SH2 domains (also referred to herein as Src homology domain 2 or Src homology-2).

Generally, kinases are enzymes known to regulate the majority of cellular pathways, especially pathways involved in signal transduction or the transmission of signals within a cell. Because protein kinases have profound effect on a cell, kinase activity is highly regulated. Kinases can be turned on or off by phosphorylation (sometimes by the kinase itself—cis-phosphorylation/autophosphorylation) and by binding to activator proteins, inhibitor proteins or small molecules.

Deregulated kinase activity is a frequent cause of disease, particularly cancer where kinases regulate many aspect that control cell growth, movement and death. For example, neoplastic transformation in which multiple genetic defects such as translocation, mutations within oncogenes and the like, have been implicated in the development of leukemia. Many of these genetic defects have been identified as key components of signaling pathways responsible for proliferation and differentiation.

The Src family of kinases, “SFKs,” are also referred to as the transforming (sarcoma inducing) gene of Rous sarcoma virus. SFKs are cytoplasmic proteins with tyrosine-specific protein kinase activity that associates with the cytoplasmic face of the plasma membrane. Silverman L., Sigal C. T., Resh M. D., Binding of pp60v-src to Membranes: Evidence for Multiple Membrane Interactions, Biochem Cell Biol. 1992 70(10-11):1187-92. There are 9 Src kinases in the human genome: v-Src, c-Src, Fyn, Yes, Fgr, Lyn, Hck, Lek, and Blk. These proteins are all closely related to each other and share the same regulatory mechanism. Brickell, P. M, The p60c-src Family of Protein-Tyrosine Kinases: Structure, Regulation, and Function, Crit. Rev Oncog. 1992; 3(4):401-46. More specifically, Src kinases are 52-62 kD proteins having six distinct functional domains: SH4 (src homology 4), a unique domain, SH3, SH2, SH1 and a C-terminal regulatory region. Brown, M. T., Cooper, J. A., Regulations, Substrates, and Functions of Src, Biochim. Biophys. Acta. 1996, 1287(2-3):121-49.

SH4 domain contains the myristylation signals that guide the Src molecule to the cell membrane. The N-terminal half of Src kinase contains the site(s) for its tyrosine phosphorylation, and phosphorylation of tyrosine (Y) 416 regulates the catalytic activity of Src. Thomas, S. M., Brugge, J. S., Cellular Functions Regulated By Src Family Kinases, Ann. Rev. Cell Dev. Biol., 1997, 13: 513-609. Because the N-terminal region of the Src kinase is myristylated, Src can be associated with the cell membrane. This domain is responsible for the specific interaction of Src with particular receptors and protein targets. Id. The C-terminal has a phosphotyrosine residue (Tyr 527).

The modulating regions, SH3 and SH2, control intra- as well as intermolecular interactions with protein substrates which affect Src catalytic activity, localization and association with protein targets. Pawson, T., Grish, G. D., SH2 and SH3 Domains: From Structure to Function, Cell, 1992, 71: 359-362. The SH3 domain recognizes polyproline helices. The kinase domain, SH1, also known as the tyrosine kinase domain and/or catalytic binding domain, is found in all proteins of the Src family and is responsible for the tryosine kinase activity. The SH 1 domain has a central role in binding of substrates.

The Src kinases (herein also referred to as: “Src family of kinases,” “Src proteins,” and “SFKs”) are normally kept off by an autoinhibitory interaction between the phosphotyrosine-binding module (SH2) that is located within the protein before the catalytic kinase domain, and its C-terminal phosphotyrosine (Tyr 527). One form of Src kinase, v-Src, encoded by Rous Sarcoma virus is, however, constitutively active. The v-src gene encodes the protein (v-Src) that on its own can induce the morphological and tumor causing potential of the virus in culture cells, and is indeed, the first of many tumor-causing genes (oncogenes) to be isolated from viruses that have normal counterparts in animal genomes. Takeya, T., Hanafusa, H. Structure and Sequence of the Cellular Gene Homologous to the RSV src Gene and the Mechanism for Generating the Transforming Virus Cell, 1983, 32: 881-890. The oncogenic properties of the v-Src protein arise from disruptions in an internal control mechanism that normally prevents the activation of the protein in the absence of external signals.

The protein encoded by the cellular counterpart of v-Src is the protein, c-Src. By contrast, the normal cellular Src, c-Src, is usually inactive until appropriately activated. Fukami, Y., Sato, K., Ikeda, K., Kamisango, K., Koizumi, K., Matsuno, T., Evidence for Autoinhibitory Regulation of the c-src Gene Product. A Possible Interaction Between the src Homology 2 Domain and Autophosphorylation Site, J. Biol. Chem., 1993 268(2), 1132-1140. c-Src participates in the signal transduction pathways of receptors that regulate cell growth in animal cell. v-Src differs from cellular Src (c-Src) on the basis of the structural differences in C-terminal region responsible for regulation of kinase activity. V-Src always exists in opened, active conformation, whereas c-Src is flexible and normally inactive. Thomas et al., Ann. Rev. Cell Dev. Biol., at 513-609. Activation of c-Src is reportedly involved in carcinoma cell migration and metastasis. Sakamoto, M., Takamura, M., Ino, Y., Miura, A., Genda, T. Hirohashi, S., Involvement of c-Src in Carcinoma Cell Motility and Metastasis, Cancer Science, 2001 92(9): 941-946.

Recently, small-molecule tyrosine kinase inhibitors have been identified as a potent inhibitor of Src kinases. In head and neck squamous carcinoma and non-small cell lung cancer cell lines, dastinib results in cytotoxicity, cell cycle arrest and apoptosis. However, despite the durable inhibition of SFKs and initial inhibition of STAT3, STAT3 is not durably inhibited.

Of the various STAT pathways, STAT3 has been identified as a mediator cell proliferation. Inhibition of SFKs does not durably inhibit STAT3. While the SFK inhibitor may initially inhibit STAT3, within a short period of time, STAT3 subsequently re-activiates and is expressed. Johnson, F. M., Saigal, B, Talpaz, M. and Donato, N. J., Dasatinib (BMS-354825) Tyrosine Kinase Inhibitor Suppresses Invasion and Induces Cell Cycle Arrest and Apoptosis of Head and Neck Squamous Cell Carcinoma and Non-Small Cell Lung Cancer Cells, Clin. Cancer Res. 11:6924-6932, 2005; Nam, S, Kim, D., Cheng, J. Q., Zhang, S., Lee, J. H., Buettner, R., Mirosevich, J., Lee, F. Y., and Jove, R., Action of the Src Family Kinase Inhibitor, Dasatinib (BMS-354825), on Human Prostate Cancer Cells, Cancer Res, 65: 9185-9189, 2005; Donato, N. J., Wu, J. Y., Stapley, J., Lin, H., Arlinghaus, R., Aggarwal, B. B., Shishodia, S., Albitar, M., Hayes, K., Kantarjian, H., and Talpaz, M., Imatinib Mesylate Resistance Through BCR-ABL Independence in Chronic Myelogenous Leukemia, Cancer Res, 64: 672-677, 2004; and Hambek, M., Baghi, M., Strebhardt, K., May, A., Adunka, O., Gstottner, W., and Knecht, R., STAT 3 Activation in Head and Neck Squamous Cell Carcinomas is Controlled by the EGFR, Anticancer Res, 24: 3881-3886, 2004.

The STAT (Signal Transducers and Activators of Transcription) proteins are transcription factors specifically activated to regulate gene transcription when cells encounter cytokines and growth factors. STAT proteins act as signal transducers in the cytoplasm and transcription activators in the nucleus. Kisseleva T., Bhattacharya S., Braunstein J., Schindler C. W., Signaling Through the JAK/STAT Pathway, Recent Advances and Future Challenges, Gene 285: 1-24 (2002).

STAT proteins regulate many aspects of cell growth, survival and differentiation. Quadros, M. R., Peruzzi, F., Kari, C., and Rodeck, U., Complex Regulation of Signal Transducers and Activators of Transcription 3 Activation in Normal and Malignant Keratinocytes, Cancer Res, 64: 3934-3939, 2004. The seven mammalian STAT family members identified are: STAT1, STAT2, STAT3, STAT4, STAT5a, STAT5b and STATE.

STAT proteins play a critical role in regulating innate and acquired host immune responses. Dysregulation of at least two STAT signaling cascades (i.e. Stat3 and Stat5) is associated with cellular transformation. Bromberg, J., Darnell, J. E. Jr., The Role of STATs in Transcriptional Control and Their Impact on Cellular Function, Oncogene, 2000, 19(21): 2468-2473. The seven STAT proteins identified in mammals range in size from 750 and 850 amino acids. The chromosomal distribution of these STATs, as well as the identification of STATs in more primitive eukaryotes, suggest that this family arose from a single primordial gene.

STAT3 can be activated by growth factor receptors, cytokine receptors and non-receptor tyrosine kinases. As reported, STAT3 activation mediated by EGFR, EPO-R, and IL-6 R via c-Src or JAK2. See e.g., Lai, S. Y., Childs, E. E., Xi, S., Coppelli, F. M., Gooding, W. E., Wells, A., Ferris, R. L., and Grandis, J. R., Erythropoietin-Mediated Activation of JAK-STAT Signaling Contributes to Cellular Invasion in Head and Neck Squamous Cell Carcinoma, Oncogene, 24: 4442-4449, 2005; Siavash, H., Nikitakis, N. G., and Sauk, J. J., Abrogation of IL-6-Mediated JAK Signalling by the Cyclopentenone Prostaglandin 15d-PGJ(2) in Oral Squamous Carcinoma Cells, Br J Cancer, 91: 1074-1080, 2004; & Quadros, M. R., Peruzzi, F., Kari, C., and Rodeck, U., Complex Regulation of Signal Transducers and Activators of Transcription 3 Activation in Normal and Malignant Keratinocytes, Cancer Res, 64: 3934-3939, 2004. MAPK activation can lead to decreased STAT3 phosphorylation. In solid tumors, PDGFR and c-Met can also activate STAT3 via c-Src. IGFR1 and EGFR can active STAT3 in a JAK-independent manner. STAT3 activation can lead to activation of several downstream target genes including Bcl-XL, cyclin D1 and VEGF.

STATs share structurally and functionally conserved domains including: an N-terminal domain that strengthens interactions between STAT dimers on adjacent DNA-binding sites; a coiled-coil STAT domain that is implicated in protein-protein interactions; a DNA-binding domain with an immunoglobulin-like fold similar to p53 tumor suppressor protein; an EF-hand-like linker domain connecting the DNA-binding and SH2 domains; an SH2 domain that acts as a phosphorylation-dependent switch to control receptor recognition and DNA-binding; and a C-terminal transactivation domain. Chen X., Vinkemeier U., Zhao Y., Jeruzalmi D., Darnell J. E., Kuriyan J., Crystal Structure of a Tyrosine Phosphorylated STAT-1 Dimer Bound to DNA, Cell 93: 827-839 (1998).

STAT signaling has been implicated in various cancers. Song, J. I. and Grandis, J. R., STAT Signaling in Head and Neck Cancer, Oncogene, 19: 2489-2495, 2000. In particular, STAT3 is tyrosine-phosphorylated and activated by a number of kinases. STAT3 activation is known to abrogate growth factor dependence which contributes to certain carcinoma tumor growth. Kijima, T., Niwa, H., Steinman, R. A., Drenning, S. D., Gooding, W. E., Wentzel, A. L., Xi, S., and Grandis, J. R., STAT3 Activation Abrogates Growth Factor Dependence And Contributes To Head And Neck Squamous Cell Carcinoma Tumor Growth In Vivo, Cell Growth Differ, 13: 355-362, 2002. Activation of STAT3 is also reported to regulate survival in human non-small cell carcinoma cells. Song, L., Turkson, J., Karras, J. G., Jove, R., and Haura, E. B., Activation Of Stat3 By Receptor Tyrosine Kinases And Cytokines Regulates Survival In Human Non-Small Cell Carcinoma Cells, Oncogene, 22: 4150-4165, 2003.

Disorders or conditions where a method of identifying the reactivation of STAT3 in a subject may be useful after the administration of an SFK (STAT-associated disorder) inhibitor include cancer, such as colorectal cancer, and cancer of the breast, lung, prostate, bladder, cervix and skin. More specifically, the neoplasias that may be identified by the use a STAT3 reactivation biomarker include, but not limited to, brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. The neoplasia can further be selected from gastrointestinal cancer, liver cancer, bladder cancer, pancreas cancer, ovary cancer, prostate cancer, cervical cancer, lung cancer, breast cancer and skin cancer, such as squamous cell and basal cell cancers.

SFK inhibitors have been developed that exhibit favorable pharmacokinetics when administered orally to humans and appear tolerated in humans without severe hemtologic or bone toxicity. As mentioned above, one such inhibitor is dastinib, a thiazole-based dual SFK/Abl inhibitor. A wide variety of SFK inhibitors may be useful in the practice of the subject invention. The following examples are not intended to be exhaustive. Dasatinib, available from Bristol-Myers Squibb Company, Wallingford, Conn., is a small molecule inhibitor. U.S. Pat. No. 6,723,694 incorporated herein by reference discloses other SFKs modulators. U.S. Pat. No. 6,610,688, incorporated herein by reference, teaches 4-substituted 7-aza-indolin-2-ones that are inhibitors of c-src. Similarly, U.S. Pat. No. 6,455,270 incorporated herein by reference teaches lichen-derived organic acids such as vulpinic acid and usnic acid which have been found to be effective inhibitors of eukaryotic protein kinase activity, including c-src. U.S. Pat. No. 6,150,359 incorporated herein by reference teaches naphthyridinones that inhibit protein tyrosine kinase and cell cycle kinase mediated cellular proliferation that may be useful in the practice of the disclosed inventions. More recently, U.S Published Patent Application, US20060258642, incorporated herein by reference, teaches quinazoline derivatives have been used for the treatment of tumors. The target kinase disclosed in this recent published application is the Src family kinases, especially c-Src. PP2 (4-amino-5-(4-chlorophenyl)-7-(t-butyl)pyrazolo[3,4-d]pyrimidine) is a potent, Src family-selective tyrosine kinase inhibitor. Hanke, J. H. et al. 1996: J Biol Chem 271, 695-701. Other Src inhibitors that may be used are taught in US Patent Applications including US20060074094, US20060058341, US20060035897, US20060004043, US20050153955, US20040186157, and US20040072836, each of which is incorporated by reference.

Dasatinib can also be referred to as BMS-354825, and N-(2-chloro-6-methylphenyl)-2-((6-(4-(2-hydroxyethyl)-1-piperazinyl)-2-methyl-4-pyrimidinyl)amino)-1,3-thiazole-5-carboxamide in accordance with IUPAC nomenclature. Use of the term “N-(2-chloro-6-methylphenyl)-2-[[6-[4-(2-hydroxyethyl)-1-piperazinyl]-2-methyl-4-pyrimidinyl]amino]-5-thiazolecarboxamide” encompasses (unless otherwise indicated) solvates (including hydrates) and polymorphic forms of the compound or its salts (such as the monohydrate form described in US 20060004067A1 at pages 25-28, incorporated herein by reference in its entirety and for all purposes). Pharmaceutical compositions of N-(2-chloro-6-methylphenyl)-2-[[6-[4-(2-hydroxyethyl)-1-piperazinyl]-2-methyl-4-pyrimidinyl]amino]-5-thiazolecarboxamide include all pharmaceutically acceptable compositions comprising N-(2-chloro-6-methylphenyl)-2-[[6-[4-(2-hydroxyethyl)-1-piperazinyl]-2-methyl-4-pyrimidinyl]amino]-5-thiazolecarboxamide and one or more diluents, vehicles and/or excipients, such as those compositions described in U.S. Ser. No. 11/402,502, filed Apr. 12, 2006, incorporated herein by reference in its entirety and for all purposes. One example of a pharmaceutical composition comprising N-(2-chloro-6-methylphenyl)-2-[[6-[4-(2-hydroxyethyl)-1-piperazinyl]-2-methyl-4-pyrimidinyl]amino]-5-thiazolecarboxamide is SPRYCEL® (Bristol-Myers Squibb Company). SPRYCEL® comprises N-(2-chloro-6-methylphenyl)-2-[[6-[4-(2-hydroxyethyl)-1-piperazinyl]-2-methyl-4-pyrimidinyl]amino]-5-thiazolecarboxamide as the active ingredient, also referred to as dasatinib, and as inactive ingredients or excipients, lactose monohydrate, microcrystalline cellulose, croscarmellose sodium, hydroxypropyl cellulose, and magnesium stearate in a tablet comprising hypromellose, titanium dioxide, and polyethylene glycol.

As is known in the art, N-(2-chloro-6-methylphenyl)-2-[[6-[4-(2-hydroxyethyl)-1-piperazinyl]-2-methyl-4-pyrimidinyl]amino]-5-thiazolecarboxamide refers to a compound having the following structure (I):

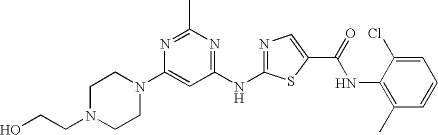

The biomarkers disclosed herein may be useful to identify an individual suffering from STAT3 reactivation can comprise the steps of determining whether a biological sample obtained from the individual comprises phosphorylated STAT3, wherein the presence of phosphorylated STAT3 is indicative of the individual being at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, and administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, sufficient to treat the individual. The therapeutically effective amount will depend upon whether or not the individual has STAT3 reactivation and whether or not the therapy with N-(2-chloro-6-methylphenyl)-2-[[6-[4-(2-hydroxyethyl)-1-piperazinyl]-2-methyl-4-pyrimidinyl]amino]-5-thiazolecarboxamide will be combined with a second therapy. Currently, the recommended dosage for N-(2-chloro-6-methylphenyl)-2-[[6-[4-(2-hydroxyethyl)-1-piperazinyl]-2-methyl-4-pyrimidinyl]amino]-5-thiazolecarboxamide is twice daily as a 70 mg tablet or 100 mg once daily referred to as SPRYCEL™. In certain embodiments, if an individual is determined to have STAT3 reactivation that renders cells partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide treatment, the dosage of the drug can be increased. Alternatively, the drug can be administered in combination with a second therapy for treating the STAT3 reactivation associated disorder. The second therapy can be any therapy effective in treating the disorder, including, for example, therapy with a JAK kinase inhibitor, including, but not limited to, AG490 or pyridone 6; another protein kinase inhibitor such as imatinib, AMN107, PD180970, GGP76030, AP23464, SKI 606, NS-187, and/or AZD0530; therapy with a tubulin stabilizing agent for example, pacitaxol, epothilone, taxane, and the like; therapy with an ATP non-competitive inhibitor such as ONO12380; therapy with an Aurora kinase inhibitor such as VX-680; therapy with a p38 MAP kinase inhibitor such as BIRB-796; or therapy with a farnysyl transferase inhibitor. The dosage of N-(2-chloro-6-methylphenyl)-2-[[6-[4-(2-hydroxyethyl)-1-piperazinyl]-2-methyl-4-pyrimidinyl]amino]-5-thiazolecarboxamide treatment or a pharmaceutically acceptable salt, hydrate, or solvate thereof can remain the same, be reduced, or be increased when combined with a second therapy.

The methods of treating a STAT3 reactivation associated disorder in an individual suffering from cancer, will ideally inhibit proliferation of cancerous cells and/or induce apoptosis of the cancerous cells.

The individual to be screened or treated by the methods herein can be one that has received administration of a first kinase inhibitor to which the cancer cells in said individual have become resistant or at least partially resistant. The kinase inhibitor can be imatinib, N-(2-chloro-6-methylphenyl)-2-[[6-[4-(2-hydroxyethyl)-1-piperazinyl]-2-methyl-4-pyrimidinyl]amino]-5-thiazolecarboxamide, another kinase inhibitor, or any combination thereof. Alternatively, the individual will have not yet had treatment with a protein kinase inhibitor.

Combinations treatments comprising a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are described in U.S. Ser. No. 10/886,955, filed Jul. 8, 2004, U.S. Ser. No. 11/265,843, filed Nov. 3, 2005, and U.S. Ser. No. 11/418,338, filed May 4, 2006, each of which are incorporated herein by reference in their entirety and for all purposes.

Methods of establishing a treatment regimen for an individual having a STAT3 reactivation related disorder are provided herein. The treatment regimen can comprise the administration of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, at a higher dose or dosing frequency than recommended for an individual not having STAT3 reactivation. Alternatively, the treatment regiment can comprise combination therapy with N-(2-chloro-6-methylphenyl)-2-[[6-[4-(2-hydroxyethyl)-1-piperazinyl]-2-methyl-4-pyrimidinyl]amino]-5-thiazolecarboxamide and any other agent that works to inhibit proliferation of cancerous cells or induce apoptosis of cancerous cells, including, for example, a JAK inhibitor, including but not limited to, AG490 or pyridone 6; a tubulin stabilizing agent, a farnysyl transferase inhibitor, a BCR-ABL T315I inhibitor and/or another protein tyrosine kinase inhibitor. Preferred other agents include imatinib, AMN107, PD180970, CGP76030, AP23464, SKI 606, NS-187, or AZD0530. Also included are ATP non-competitive inhibitors, including, for example, ON012380, Aurora kinase inhibitors, including, fore example, VX-680, and p38 MAP kinase inhibitors, including, for example, BIRB-796. The treatment regimen can include administration of a higher dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent, a reduced dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent, or an unchanged dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent.

### Exemplary Indications, Conditions, Diseases, and Disorders

The methods of determining responsiveness of an individual having a STAT3 reactivation associated disorder to a certain treatment regimen and methods of treating an individual having a STAT3 reactivation associated disorder are provided herein.

The term “STAT3 reactivation” as used herein relates to the phosphorylated status of STAT3 and STAT3 DNA binding. There is no change in total STAT3 protein levels.

“STAT3 reactivation associated disorders” are disorders where STAT3 is reactivated but total levels are not affected. As such, PCR is not be an appropriate method to measure reactivation of STAT3. Experimentally, STAT3 activation can be measured in two ways: 1. Phosphorylation that can be measured by Western blotting and 2. DNA binding that can be measured by ELISA or EMSA assays.

The disclosed biomarker may be useful in connection with disorders such as lung cancer, leukemias, including, for example, chronic myeloid leukemia, acute lymphoblastic leukemia, and Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is T-ALL, chronic myeloid leukemia (CML), Ph+ ALL, AML, imatinib-resistant CML, imatinib-intolerant CML, accelerated CML, lymphoid blast phase CML,

A “solid tumor” includes, for example, sarcoma, melanoma, carcinoma, or other solid tumor cancer.

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, chronic myeloid leukemia, acute lymphoblastic leukemia, and/or Philadelphia chromosome positive acute lymphoblastic leukemia (Ph+ ALL) are all diseases.

“STAT3 reactivation associated disorder” is used to describe a STAT3 reactivation associated disorder in which the cells involved in said disorder continue to proliferate on account of the STAT3 reactivation. Treatment of such a condition will require a compound that is at least partially effective against the STAT3 reactivation. The inventors discovered that after treatment with N-(2-chloro-6-methylphenyl)-2-[[6-[4-(2-hydroxyethyl)-1-piperazinyl]-2-methyl-4-pyrimidinyl]amino]-5-thiazolecarboxamide, certain cells developed STAT3 reactivation despite initially showing STAT3 inhibition. This disclosure provides, among other things, methods of identifying if an individual has a STAT3 reactivation associated disorder.

The methods of treating disorders resulting from “imatinib-resistant mutations” in the BCR-ABL kinase, which may be pre-existing to STAT3 reactivation, or may appear during or after STAT3 reactivation are also provided.

“Imatinib-resistant mutation” refers to a specific mutation in the amino acid sequence of BCR-ABL that confers upon cells that express said mutation resistance to treatment with imatinib. Mutations that may render a BCR-ABL protein at least partially imatinib resistant can include, for example, E279K, F359C, F359I, L3641, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, E316G, F317L, M318T, Y320C, Y320H, G321E, D325H, Y326C, L327P, R328K, E329V, Q333L, A337V, V339G, L342E, M343V, M343T, A344T, A344V, 1347V, A350T, M351T, E352A, E352K, E355G, K357E, N358D, N358S, F359V, F359C, F359I, I360K, I360T, L364H, L3641, E373K, N374D, K378R, V379I, A380T, A380V, D381G, F382L, L387M, M388L, T389S, T392A, T394A, A395G, H396K, H396R, A399G, P402T, T406A, S417Y, and F486S (see, for example, U.S. Publication Number 20030158105, incorporated herein by reference in its entirety and for all purposes).

The methods of treating disorders resulting from “N-(2-chloro-6-methylphenyl)-2-[[6-[4-(2-hydroxyethyl)-1-piperazinyl]-2-methyl-4-pyrimidinyl]amino]-5-thiazolecarboxamide-resistant mutations” in the BCR-ABL kinase are taught herein, which may be pre-existing to STAT3 reactivation, or may appear during or after STAT3 reactivation.

“N-(2-chloro-6-methylphenyl)-2-[[6-[4-(2-hydroxyethyl)-1-piperazinyl]-2-methyl-4-pyrimidinyl]amino]-5-thiazolecarboxamide-resistant BCR-ABL mutation” refers to a specific mutation in the amino acid sequence of BCR-ABL that confers upon cells that express said mutation at least partially resistance to treatment with N-(2-chloro-6-methylphenyl)-2-[[6-[4-(2-hydroxyethyl)-1-piperazinyl]-2-methyl-4-pyrimidinyl]amino]-5-thiazolecarboxamide. Such mutations may include the F317V, F317I, F317H, T315I, and T315A mutations. Other mutations are disclosed in PCT Publication No. WO2007/011765, filed Jul. 13, 2006; PCT Publication No. WO2007/065124, filed Nov. 30, 2006; PCT Publication No. WO2007/056177, filed Nov. 3, 2006; and PCT Publication No. WO2007/109527, filed Mar. 16, 2007; and are hereby incorporated by reference in their entirety and for all purposes.

“Imatinib-resistant CML” refers to a CML in which the cells involved in CML are resistant to treatment with imatinib. Generally it is a result of a mutation in BCR-ABL.

“Imatinib-intolerant CML” refers to a CML in which the individual having the CML is intolerant to treatment with imatinib, i.e., the toxic and/or detrimental side effects of imatinib outweigh any therapeutically beneficial effects.

### Treatment Regimens

The invention encompasses treatment methods based upon the demonstration that patients with STAT3 reactivation may have varying degrees of resistance and/or sensitivity to N-(2-chloro-6-methylphenyl)-2-[[6-[4-(2-hydroxyethyl)-1-piperazinyl]-2-methyl-4-pyrimidinyl]amino]-5-thiazolecarboxamide, and/or imatinib. Thus the methods disclosed herein can be used, for example, in determining whether or not to treat an individual with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof; whether or not to treat an individual with a more aggressive dosage regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof; or whether or not to treat an individual with combination therapy, i.e., a combination of tyrosine kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and JAK inhibitors, AG490 or pyridone 6; and/or additional STAT3 reactivation inhibitors(s) (e.g., such as imatinib, AMN107, PD180970, GGP76030, AP23464, SKI 606, NS-187, and/or AZD0530); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and a tubulin stabilizing agent (such as, for example, pacitaxol, epothilone, taxane, and the like.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and a farnysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and ATP non-competitive inhibitors ONO12380; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and Aurora kinase inhibitor VX-680; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and p38 MAP kinase inhibitor BIRB-796; any other combination disclosed herein.

The terms “treating”, “treatment” and “therapy” as used herein refer to curative therapy, prophylactic therapy, preventative therapy, and mitigating disease therapy.

For use herein, a BCR-ABL inhibitor refers to any molecule or compound that can partially inhibit BCR-ABL or mutant BCR-ABL activity or expression. These include inhibitors of the Src family kinases such as BCR/ABL, ABL, c-Src, SRC/ABL, and other forms including, but not limited to, JAK, FAK, FPS, CSK, SYK, and BTK. A series of inhibitors, based on the 2-phenylaminopyrimidine class of pharmacophotes, has been identified that have exceptionally high affinity and specificity for Abl (see, e.g., Zimmerman et al., Bloorg, Med. Chem. Lett. 7, 187 (1997)). All of these inhibitors are encompassed within the term a BCR-ABL inhibitor. Imatinib, one of these inhibitors, also known as STI-571 (formerly referred to as Novartis test compound CGP 57148 and also known as Gleevec), has been successfully tested in clinical trail a therapeutic agent for CML. AMN107, is another BCR-ABL kinase inhibitor that was designed to fit into the ATP-binding site of the STAT3 reactivation protein with higher affinity than imatinib. In addition to being more potent than imatinib (IC50<30 nM) against wild-type BCR-ABL, AMN107 is also significantly active against 32/33 imatinib-resistant BCR-ABL mutants. In preclinical studies, AMN107 demonstrated activity in vitro and in vivo against wild-type and imatinib-resistant BCR-ABL-expressing cells. In phase I/II clinical trials, AMN107 has produced haematological and cytogenetic responses in CML patients, who either did not initially respond to imatinib or developed imatinib resistance (Weisberg et al., British Journal of Cancer (2006) 94, 1765-1769, incorporated herein by reference in its entirety and for all purposes). SKI-606, NS-187, AZD0530, PD180970, CGP76030, and AP23464 are all examples of kinase inhibitors that can be used for treatments. SKI-606 is a 4-anilino-3-quinolinecarbonitrile inhibitor of Abl that has demonstrated potent antiproliferative activity against CML cell (Golas et al., Cancer Research (2003) 63, 375-381). AZD0530 is a dual Abl/Src kinase inhibitor that is in ongoing clinical trials for the treatment of solid tumors and leukemia (Green et al., Preclinical Activity of AZD0530, a novel, oral, potent, and selective inhibitor of the Src family kinases. Poster 3161 presented at the EORTC-NCI-AACR, Geneva Switzerland 28 Sep. 2004). PD180970 is a pyrido[2,3-d]pyrimidine derivative that has been shown to inhibit BCR-ABL and induce apoptosis in BCR-ABL expressing leukemic cells (Rosee et al., Cancer Research (2002) 62, 7149-7153). CGP76030 is dual-specific Src and Abl kinase inhibitor shown to inhibit the growth and survival of cells expressing imatinib-resistant BCR-ABL kinases (Warmuth et al., Blood, (2003) 101(2), 664-672). AP23464 is an ATP-based kinase inhibitor that has been shown to inhibit imatinib-resistant BCR-ABL mutants (O′Hare et al., Clin Cancer Res (2005) 11(19), 6987-6993). NS-187 is a selective dual Bcr-Abl/Lyn tyrosine kinase inhibitor that has been shown to inhibit imatinib-resistant BCR-ABL mutants (Kimura et al., Blood, 106(12):3948-3954 (2005)).

A “farnysyl transferase inhibitor” can be any compound or molecule that inhibits farnysyl transferase. The farnysyl transferase inhibitor can have formula (II), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt. The compound of formula (II) is a cytotoxic FT inhibitor which is known to kill non-proliferating cancer cells preferentially. The compound of formula (II) can further be useful in killing stem cells.

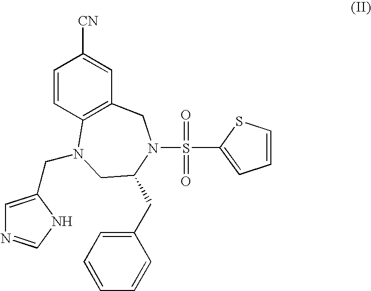

The compound of formula (II), its preparation, and uses thereof are described in U.S. Pat. No. 6,011,029, which is herein incorporated by reference in its entirety and for all purposes. Uses of the compound of formula (II) are also described in WO2004/015130, published Feb. 19, 2004, which is herein incorporated by reference in its entirety and for all purposes.

For use herein, combination therapy refers to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof with a second therapy at such time that both the second therapy and N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, will have a therapeutic effect. Such administration can involve concurrent (i.e., at the same time), prior, or subsequent administration of the second therapy with respect to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof.

Treatment regimens can be established based upon the presence of STAT3 reactivation, and potentially, in addition to, one or more mutant BCR-ABL kinases disclosed herein. For example, the invention encompasses screening cells from an individual who may suffer from, or is suffering from, a disorder that is commonly treated with a kinase inhibitor. Such a disorder can include myeloid leukemia or disorders associated therewith, or cancers described herein. The cells of an individual are screened, using methods known in the art, for identification of a mutation in a BCR-ABL kinase. Mutations of interest are those that result in BCR-ABL kinase being constitutively activated. Specific mutations may include, for example, F317I (wherein the phenylalanine at position 317 is replaced with an isoleucine), and T315A (wherein the threonine at position 315 is replaced with an alanine). Other mutations include, for example, E279K, F359C, F359I, L3641, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, 1293T, P296S, L298M, L298P, V299L, Q300R, G303E, V304A, V304D, C305S, C305Y, T306A, F311L, 1314V, T315I, E316G, F317L, M318T, Y320C, Y320H, G321E, D325H, Y326C, L327P, R328K, E329V, Q333L, A337V, V339G, L342E, M343V, M343T, A344T, A344V, 1347V, A350T, M351T, E352A, E352K, E355G, K357E, N358D, N358S, F359V, F359C, F359I, I360K, I360T, L364H, L3641, E373K, N374D, K378R, V379I, A380T, A380V, D381G, F382L, L387M, M388L, T389S, T392A, T394A, A395G, H396K, H396R, A399G, P402T, T406A, S417Y, F486S or any combination thereof, i.e., M244V, G250E, Q252H, Q252R, Y253F, Y253H, E255K, E255V, T3151, F317L, M351T, E355G, F359V, H396R, F486S and any combination thereof; M244V, E279K, F359C, F359I, L364I, L387M, F486S and any combination thereof; and L248R, Q252H, E255K, V299L, T315I, F317V, F317L, F317S and any combination thereof.

If an activating BCR-ABL kinase mutation is found in the cells from said individual, treatment regimens can be developed appropriately. For example, if STAT3 reactivation is present, in the absence of a BCR-ABL mutation, appropriate treatment may merely require administering a pharmaceutically acceptable dose of N-(2-chloro-6-methylphenyl)-2-[[6-[4-(2-hydroxyethyl)-1-piperazinyl]-2-methyl-4-pyrimidinyl]amino]-5-thiazolecarboxamide with a JAK inhibitor. Alternatively, if STAT3 reactivation is present in addition to a BCR-ABL mutation, such an identified mutation can indicate that said cells are or will become at least partially resistant to commonly used kinase inhibitors. For example, a F317I or T315A mutation can indicate that the cells in an individual are or are expected to become at least partially resistant to treatment with a kinase inhibitor such as N-(2-chloro-6-methylphenyl)-2-[[6-[4-(2-hydroxyethyl)-1-piperazinyl]-2-methyl-4-pyrimidinyl]amino]-5-thiazolecarboxamide. As disclosed herein, in such cases, treatment can include the use of an increased dosing frequency or increased dosage of N-(2-chloro-6-methylphenyl)-2-[[6-[4-(2-hydroxyethyl)-1-piperazinyl]-2-methyl-4-pyrimidinyl]amino]-5-thiazolecarboxamide or a salt, hydrate, or solvate thereof, a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and another kinase inhibitor drug such as imatinib, AMN107, PD180970, GGP76030, AP23464, SKI 606, and/or AZD0530; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor; any other combination disclosed herein; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein. In one aspect, an increased level of N-(2-chloro-6-methylphenyl)-2-[[6-[4-(2-hydroxyethyl)-1-piperazinyl]-2-methyl-4-pyrimidinyl]amino]-5-thiazolecarboxamide would be about 10, 20, 30, 40, 50, 60, 70, 80, 90, or 95% more than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual, or about 1.5×, 2×, 2.5×, 3×, 3.5×, 4×, 4.5×, 5×, 6×, 7×, 8×, 9×, or 10× more N-(2-chloro-6-methylphenyl)-2-[[6-[4-(2-hydroxyethyl)-1-piperazinyl]-2-methyl-4-pyrimidinyl]amino]-5-thiazolecarboxamide than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual. Alternatively, an appropriate treatment regimen for the presence of STAT3 reactivation may also require the same or similar increased dose or dose frequency of N-(2-chloro-6-methylphenyl)-2-[[6-[4-(2-hydroxyethyl)-1-piperazinyl]-2-methyl-4-pyrimidinyl]amino]-5-thiazolecarboxamide as outlined herein.

Additionally, dosage regimens can be further adapted based upon the presence of additional amino acid mutation in a BCR-ABL kinase. As described herein, a mutation in E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, 1293T, P296S, L298M, L298P, V299L, Q300R, G303E, V304A, V304D, C305S, C305Y, T306A, F311L, 1314V, T3151, E316G, F317L, M318T, Y320C, Y320H, G321E, D325H, Y326C, L327P, R328K, E329V, Q333L, A337V, V339G, L342E, M343V, M343T, A344T, A344V, 1347V, A350T, M351T, E352A, E352K, E355G, K357E, N358D, N358S, F359V, F359C, F359I, I360K, 1360T, L364H, L3641, E373K, N374D, K378R, V379I, A380T, A380V, D381G, F382L, L387M, M388L, T389S, T392A, T394A, A395G, H396K, H396R, A399G, P402T, T406A, S417Y, F486S, or any combination thereof can indicate that the BCR-ABL kinase has developed at least partial resistance to therapy with a protein kinase inhibitor such as imitinab.

A therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof can be orally administered as an acid salt of N-(2-chloro-6-methylphenyl)-2-[[6-[4-(2-hydroxyethyl)-1-piperazinyl]-2-methyl-4-pyrimidinyl]amino]-5-thiazolecarboxamide. The actual dosage employed can be varied depending upon the requirements of the patient and the severity of the condition being treated. Determination of the proper dosage for a particular situation is within the skill of the art. The effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof (and Compound I salt) can be determined by one of ordinary skill in the art, and includes exemplary dosage amounts for an adult human of from about 0.05 to about 100 mg/kg of body weight of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, per day, which can be administered in a single dose or in the form of individual divided doses, such as from 1, 2, 3, or 4 times per day. In certain embodiments, N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof is administered 2 times per day at 70 mg. Alternatively, it can be dosed at, for example, 50, 70, 90, 100, 110, or 120 BID, or 100, 140, or 180 once daily. It will be understood that the specific dose level and frequency of dosing for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders. The same also applies to Compound II or any combination of Compound I and II, or any combination disclosed herein.

A method of determining the responsiveness of an individual suffering from a protein tyrosine kinase-associated disorder to a combination of kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, is disclosed herein. For example, an individual can be determined to be a positive responder (or cells from said individual would be expected to have a degree of sensitivity) to a certain kinase inhibitor based upon the presence of a mutant BCR-ABL kinase. Cells that exhibit certain mutations at amino acid positions 315 and 317 of BCR-ABL kinase, for example, can develop at least partial resistance to of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof. Therefore, individuals suffering from a protein tyrosine kinase-associated disorder whose cells exhibit such a mutation are or would be expected to be partially negative responders to a particular treatment regimen with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof but a positive responder to a more aggressive treatment regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof or to combination therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and imatinib or other therapy.

A treatment regimen is a course of therapy administered to an individual suffering from a protein kinase associated disorder that can include treatment with one or more kinase inhibitors, as well as other therapies such as radiation and/or other agents (i.e., combination therapy). When more than one therapy is administered, the therapies can be administered concurrently or consecutively (for example, more than one kinase inhibitor can be administered together or at different times, on a different schedule). Administration of more than one therapy can be at different times (i.e., consecutively) and still be part of the same treatment regimen. As disclosed herein, for example, cells from an individual suffering from a protein kinase associated disorder can be found to develop at least partial resistance to N-(2-chloro-6-methylphenyl)-2-[[6-[4-(2-hydroxyethyl)-1-piperazinyl]-2-methyl-4-pyrimidinyl]amino]-5-thiazolecarboxamide. Based upon the present discovery that such cells can be sensitive to combination therapy or a more aggressive dosage or dosing regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof; a treatment regimen can be established that includes treatment with the combination either as a monotherapy, or in combination with a JAK inhibitor, another kinase inhibitor, or in combination with any other agent disclosed herein. Additionally, the combination can be administered with radiation or other known treatments.

Therefore, methods for establishing a treatment regimen for an individual suffering from STAT3 reactivation, a protein tyrosine kinase associated disorder or treating an individual suffering from a protein tyrosine kinase disorder with or without a BCR-ABL mutation, comprise determining whether a biological sample obtained from an individual demonstrates STAT3 reactivation, optionally determining whether the sample contains a mutation in the BCR-ABL kinase, and administering to the subject an appropriate treatment regimen based on whether the STAT3 reactivation is present, in addition to whether a BCR-ABL mutation is present, where applicable. The determination can be made by any method known in the art, for example, by screening said sample of cells for the presence of evidence of STAT3 reactivation, and/or screening said sample of cells for the presence of at least one mutation in a BCR-ABL kinase sequence or by obtaining information from a secondary source.

In practicing the many aspects of the invention herein, biological samples can be selected from many sources such as tissue biopsy (including cell sample or cells cultured therefrom; biopsy of bone marrow or solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample can be used, or a lysate of a cell sample can be used. In certain embodiments, the biological sample is a tissue biopsy cell sample or cells cultured therefrom, for example, cells removed from a solid tumor or a lysate of the cell sample. In certain embodiments, the biological sample comprises blood cells.

Useful pharmaceutical compositions can include compositions comprising N-(2-chloro-6-methylphenyl)-2-[[6-[4-(2-hydroxyethyl)-1-piperazinyl]-2-methyl-4-pyrimidinyl]amino]-5-thiazolecarboxamide, or a combination with a JAK inhibitor, or a combination of inhibitors of a mutant BCR-ABL kinase in an effective amount to achieve the intended purpose. The determination of an effective dose of a pharmaceutical composition of the invention is well within the capability of those skilled in the art. A therapeutically effective dose refers to that amount of active ingredient which ameliorates the symptoms or condition. Therapeutic efficacy and toxicity can be determined by standard pharmaceutical procedures in cell cultures or experimental animals, for example the ED50 (the dose therapeutically effective in 50% of the population) and LD50 (the dose lethal to 50% of the population).

Dosage regimens involving N-(2-chloro-6-methylphenyl)-2-[[6-[4-(2-hydroxyethyl)-1-piperazinyl]-2-methyl-4-pyrimidinyl]amino]-5-thiazolecarboxamide useful in practicing the invention are described in U.S. Ser. No. 10/395,503, filed Mar. 24, 2003; and Blood (ASH Annual Meeting Abstracts) 2004, Volume 104: Abstract 20, “Hematologic and Cytogenetic Responses in imatinib-Resistant Accelerated and Blast Phase Chronic Myeloid Leukemia (CML) Patients Treated with the Dual SRC/ABL Kinase Inhibitor N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study.”, by Moshe Talpaz, et al; which are hereby incorporated herein by reference in their entirety and for all purposes.

A “therapeutically effective amount” of an inhibitor of STAT3 reactivation and/or mutant BCR-ABL related disorder may be any one of the regimens outlined herein, or otherwise known in the art or as determined by the skilled artisan. However, such a “therapeutically effective amount” for STAT3 reactivation kinase may be a function of the BCR-ABL mutation present within a sample, when applicable, particularly when a “N-(2-chloro-6-methylphenyl)-2-[[6-[4-(2-hydroxyethyl)-1-piperazinyl]-2-methyl-4-pyrimidinyl]amino]-5-thiazolecarboxamide-resistant BCR-ABL mutation” is present, and may, in some circumstances depend upon when an “Imatinib-resistant BCR-ABL mutation is present. For example Shah et al disclose that cell lines with certain mutations in BCR-ABL kinase are more sensitive to N-(2-chloro-6-methylphenyl)-2-[[6-[4-(2-hydroxyethyl)-1-piperazinyl]-2-methyl-4-pyrimidinyl]amino]-5-thiazolecarboxamide than cell lines with different BCR-ABL kinase mutations. As disclosed therein, cells comprising a F317L mutation in STAT3 reactivation kinase requires three to five-fold higher concentration of N-(2-chloro-6-methylphenyl)-2-[[6-[4-(2-hydroxyethyl)-1-piperazinyl]-2-methyl-4-pyrimidinyl]amino]-5-thiazolecarboxamide than cell lines expressing a Q252R mutation. One skilled in the art will appreciate the difference in sensitivity of the mutant BCR-ABL kinase cells and determine a therapeutically effective dose accordingly.

Examples of predicted therapeutically effective doses of N-(2-chloro-6-methylphenyl)-2-[[6-[4-(2-hydroxyethyl)-1-piperazinyl]-2-methyl-4-pyrimidinyl]amino]-5-thiazolecarboxamide that may be warranted based upon the relative sensitivity of BCR-ABL kinase mutants to N-(2-chloro-6-methylphenyl)-2-[[6-[4-(2-hydroxyethyl)-1-piperazinyl]-2-methyl-4-pyrimidinyl]amino]-5-thiazolecarboxamide compared to wild-type BCR-ABL kinase can be determined using various in vitro biochemical assays including cellular proliferation, BCR-ABL tyrosine phosphorylation, peptide substrate phosphorylation, and/or autophosphorylation assays. For example, approximate therapeutically effective doses of N-(2-chloro-6-methylphenyl)-2-[[6-[4-(2-hydroxyethyl)-1-piperazinyl]-2-methyl-4-pyrimidinyl]amino]-5-thiazolecarboxamide can be calculated based upon multiplying the typical dose with the fold change in sensitivity in anyone or more of these assays for each BCR-ABL kinase mutant. O′Hare et al. (Cancer Research, 65(11):4500-5 (2005), which is hereby incorporated by reference in its entirety and for all purposes) performed analysis of the relative sensitivity of N-(2-chloro-6-methylphenyl)-2-[[6-[4-(2-hydroxyethyl)-1-piperazinyl]-2-methyl-4-pyrimidinyl]amino]-5-thiazolecarboxamide with several clinically relevant STAT3 reactivation Kinase mutants. For example, the E255V mutant had a fold change of “1” in the GST-Abl kinase assay, whereas this same mutant had a fold change of “14” in the cellular proliferation assay. Thus, a therapeutically relevant dose of N-(2-chloro-6-methylphenyl)-2-[[6-[4-(2-hydroxyethyl)-1-piperazinyl]-2-methyl-4-pyrimidinyl]amino]-5-thiazolecarboxamide for patients harboring this mutation could range, for example, anywhere from 1 to 14 fold higher than the typical dose. Accordingly, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide for any of the BCR-ABL kinase mutants can be, for example, 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 folder higher than the prescribed dose. Alternatively, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide can be, for example, 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01× of the prescribed dose. Latter dosing regimens to treat of STAT3 reactivation disorders are provided.

According to O'hare et al., the M244V mutant had a fold change of “1.3” in the GST-Abl kinase assay, a fold change of “1.1” in the autophosphorylation assay, and a fold change of “2” in the cellular proliferation assay; the G250E mutant had a fold change of “0.5” in the GST-Abl kinase assay, a fold change of “3” in the autophosphorylation assay, and a fold change of “2” in the cellular proliferation assay; the Q252H mutant had a fold change of “4” in the cellular proliferation assay; the Y253F mutant had a fold change of “0.6” in the GST-Abl kinase assay, a fold change of “4” in the autophosphorylation assay, and a fold change of “4” in the cellular proliferation assay; the Y253H mutant had a fold change of “3” in the GST-Abl kinase assay, a fold change of “2” in the autophosphorylation assay, and a fold change of “2” in the cellular proliferation assay; the E255K mutant had a fold change of “0.3” in the GST-Abl kinase assay, a fold change of “2” in the autophosphorylation assay, and a fold change of “7” in the cellular proliferation assay; the F317L mutant had a fold change of “1.5” in the GST-Abl kinase assay, a fold change of “1.4” in the autophosphorylation assay, and a fold change of “9” in the cellular proliferation assay; the M351T mutant had a fold change of “0.2” in the GST-Abl kinase assay, a fold change of “2” in the autophosphorylation assay, and a fold change of “1.4” in the cellular proliferation assay; the F359V mutant had a fold change of “0.8” in the GST-Abl kinase assay, a fold change of “2” in the autophosphorylation assay, and a fold change of “3” in the cellular proliferation assay; the H396R mutant had a fold change of “1.3” in the GST-Abl kinase assay, a fold change of “3” in the autophosphorylation assay, and a fold change of “2” in the cellular proliferation assay.

For patients harboring the T315I mutation, administration of higher doses of N-(2-chloro-6-methylphenyl)-2-[[6-[4-(2-hydroxyethyl)-1-piperazinyl]-2-methyl-4-pyrimidinyl]amino]-5-thiazolecarboxamide, or combinations of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination discloses herein; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, may be warranted. Alternatively, combinations of N-(2-chloro-6-methylphenyl)-2-[[6-[4-(2-hydroxyethyl)-1-piperazinyl]-2-methyl-4-pyrimidinyl]amino]-5-thiazolecarboxamide with a T315I inhibitor may also be warranted.

Accordingly, dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. Actual dosage levels of the active ingredients in a pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. See, e.g., the latest Remington's (Remington's Pharmaceutical Science, Mack Publishing Company, Easton, Pa.)

Many commercially available assays for kinase activity can be used to construct screens for small molecule inhibitors; such assay techniques are well known to those skilled in the art. The hits from such screens can then be profiled against arrays of other kinases to identify selective inhibitors.

### Example 1

### Identification of the Mechanism of STAT3 Reactivation

The reactivation of STAT3 after durable inhibition of SFKs is shown as a compensatory mechanism for cell survival. Experimental Design: The effect of inhibition of molecules known to be upstream of STAT3 on its reactivation was assessed with Western blotting and a quantitative bioplex phosphoprotein assay. The biological effects of SFK and JAK inhibition were assayed with an MTT assay to assess cytotoxicity and propidium iodine/annexin V staining with FACS analysis to evaluate cell cycle and apoptosis. Cytokines were quantitated using a multiplexed, particle-based FACS analysis with monoclonal antibodies to 25 known cytokines. The combination index (CI) was calculated by the Chou-Talalay equation. Results: In all cell lines, c-Src and several downstream signaling molecules (e.g. AKT, STATS, FAK) were rapidly and durably inhibited by dasatinib. However, STAT3 was initially inhibited but reactivated by 24 h in 14 solid tumor cell lines. This reactivation was observed with 3 different SFK inhibitors. We investigated several growth factor pathways known to affect STAT3 and found that its reactivation was not mediated by EGFR, IGFR, MAPK, COX2, or cytokine/growth factor release. The addition of JAK inhibitors (AG490 or pyridone 6) to dasatinib resulted in sustained inhibition of STAT3. The combination of pyridone 6 and dasatinib was synergistic in all four cell lines tested with CI that ranged from 0.09 to 0.66. The combination led to increased apoptosis. Conclusions: The reactivation of STAT3 after SFK inhibition is a compensatory pathway that allows cancer cell survival. Abrogation of this pathway using JAK inhibitors results in synergistic cytotoxicity. Given that STAT3 was reactivated in 14 of 15 solid tumor cell lines, this combination may have widespread applicability for cancer treatment.

### Example 2

### Effect of SKF Inhibition on Downstream Pathways

FIG. 2 shows the effect of SFK inhibition on downstream pathways. (A) Tu167 cells were treated with 100 nM dasatinib for the indicated times, lysed, and analyzed by Western blotting with the indicated antibodies. Dasatinib led to durable inhibition of c-Src, FAK, AKT, and STATS, but STAT3 was not durably inhibited. (B) Tu167 cells were treated with one of three different SFK inhibitors (dasatinib, PP1, or SKI606) for 24 hours then lysed, and analyzed by Western blotting with the indicated antibodies. All three SFK inhibitors led to durable c-Src inhibition but STAT3 was not inhibited at 24 hours.

### Example 3

### Identification of STAT3 after Src Inhibition

The reactivation of STAT3 diminishes the pro-apoptotic and anti-proliferative effects of SFK inhibition. We determine the biological effects of inhibiting both SFK and STAT3 in cancer.

### Materials and Methods

Materials. Dasatinib was provided by Bristol-Myers Squibb (New York, N.Y.) and was prepared as a 10 mM stock solution in DMSO. Antibodies used in Western blotting included phosphorylated MAPK (Promega, Madison, Wis.); AKT and phosphorylated AKT (New England Biolabs, Beverly, Mass.); Src (Santa Cruz Biotechnology, Santa Cruz, Calif.); pY419-c-Src, pY705-STAT3, pY694-STAT5, total EGFR, pEGFR (845, 992, 1148), pSTAT1, HIF-1-alpha, cyclin D1 (Cell Signaling Technology, Beverly, Mass.); pY861-FAK (Biosource, Camarillo, Calif.); pTyrosine (Upstate Biotechnology, Lake Placid, N.Y.); and actin (Sigma Chemical, St. Louis, Mo.). Pyridone 6, AG490, and PP1 were purchased from EMD Bioscience (La Jolla, Calif.). SKI-606 was a gift from Wyeth pharmaceuticals.

Cell Culture. Fifteen human cancer cell lines were used in this study: six HNSCC cell lines (obtained from Dr. J. Myers and Dr. G. Clayman of The University of Texas M. D. Anderson Cancer Center), four NSCLC cell lines (obtained from American Type Culture Collection, Manassas, Va.), three mesothelioma cell lines (obtained from American Type Culture Collection), and three squamous skin cancer cell lines (obtained from Dr. J. Myers). Cells were grown in monolayer cultures in Dulbecco's modified Eagle's medium (HNSCC and skin cancer cell lines) or RPMI 1640 medium (NSCLC and mesothelioma cell lines) containing 10% fetal bovine serum and 2 mM glutamine at 37° C. in a humidified atmosphere of 95% air and 5% CO₂.

Western Blot. Detached cells from each cell culture plate were collected by centrifugation, washed in PBS, and added to the cell lysate from their corresponding plates. Adherent cells were rinsed with ice-cold PBS and lysed in the cell culture plate for 20 min on ice in lysis buffer consisting of 50 mM Trizma base (ph 8; Sigma Chemical Company), 1% Triton X-100, 150 mM NaCl, 20 μg/ml leupeptin, 10 μg/ml aprotinin, 1 mM phenylmethanesulfonyl fluoride, and 1 mM sodium vanadate. Lysates were spun in a centrifuge at 14,000 rpm for 5 min, and the supernatant was collected. Equal protein aliquots were resolved by sodium dodecyl sulfate-polyacrylamide gel electrophoresis (SDS-PAGE), transferred to nitrocellulose membranes, immunoblotted with primary antibody, and detected with horseradish peroxidase-conjugated secondary antibody (BioRad Laboratories, Hercules, Calif.) and ECL reagent (Amersham Biosciences, Piscataway, N.J.).

Quantitative Bioplex Phosphoprotein Assay. Cells at 5×10⁵ per milliliter were treated with p6, dasatinib or both for 24 hours. Protein lysates were prepared by using cell lysis buffer with PMSF (Bio-Rad laboratories, Life Science Research Group, Hercules, Calif., USA) on samples collected. Phosphorylated proteins were detected by Bio-Rad phosphoprotein immunoassay kit using Bio-Plex 100 system with workstation (Bio-Rad) according to the manufacturer's protocol. The targeted phosphorylated proteins included the following: Akt (Ser473), ERK-1/2 (Thr202/Tyr204) and STAT3 (Tyr705). Briefly, 50 ml of cell lysate (adjusted to a concentration of 200-900 μg/ml of protein) was plated in the 96 well filter plate coated with anti-phospho-protein antibodies coupled beads and allowed to incubate overnight (16 hours) on a platform shaker at 300 rpm at room temperature. After vacuum-filter and washing the wells; 1 microliter of detection antibodies (25×) were added, vortexed and then incubated for 30 minutes. After additional vacuum-filter and washing of the wells, 0.5 microliter streptavidin-PE (100×) was added to each well and allowed to incubate for 10 minutes. After vacuum-filter and washing the wells, 125 microliter of resuspension buffer was added to each well and allowed to incubate for 30 seconds. Data acquisition and analysis was completed by using Bio-Plex manager (V4.1.1. software).

MTT assay. The MTT assay was used to assess cytotoxicity of drugs and drug combinations. Cells were plated into 96-well plates and incubated for 24 h using the conditions described above for standard cell culture maintenance. The cells were subsequently exposed to dasatinib, pyridone 6, or both at various concentrations for 72 h. Eight wells were treated at each concentration. After treatment, 25 μl of 3-(4,5-dimethylthiazol-2-yl)-2,5-diphenyltetrazolium (MTT) was added to each well and incubated for 3 h. The medium was then removed and 100 μL of Me₂SO was added. The absorbance of individual wells was read at 570 nM.

Determination of Synergism and Antagonism. The combination index (CI) was calculated by the Chou-Talalay equation, which takes into account both potency (Dm or IC₅₀) and the shape of the dose-effect curve. See, Chou, T. C. and Talalay, P., Quantitative Analysis Of Dose-Effect Relationships: The Combined Effects Of Multiple Drugs Or Enzyme Inhibitors, Adv Enzyme Regul, 22: 27-55, 1984; Chou, T. C., Riedeout, D., Chou, J., Bertino, J. R., and Dulbecco, R., Chemotherapeutic Synergism, Potential And Antagonism, Vol. 2, p. 371-379. San Diego, Calif.: Academic Press, 1991; Chou, T. C. and Rideout, D. C., The Median-Effect Principle And The Combination Index For Quantitation Of Synergism And Antagonism, p. 61-102. San Diego, Calif.: Academic Press, 1991. The general equation for the classic isobologram (CI=1) is given by: CI=(D)₁/(Dx)₁+(D)₂/(Dx)₂; where (Dx)₁ and (Dx)₂ in the denominators are the doses (or concentrations) for D₁ (dasatinib) and D₂ (another drug) alone that gives x % inhibition, whereas (D)₁ and (D)₂ in the numerators are the doses of dasatinib and another drug in combination that also inhibited x % (i.e., isoeffective). CI<1, CI=1, CI>1 indicate synergism, additive effect, and antagonism, respectively http://cancerres.aacrjournals.org/cgi/content/full/62/23/-B13. Nonexclusive competitors are defined as inhibitors binding to different targets or different sites of the same target. The inputs are the concentrations of single inhibitors, the combination doses at different ratios or at fixed ratios, and the fractional inhibition; ie, fraction affected (Fa) of single drugs and combinations. Fa=(Drug A control Drug A treated)/Drug A control). Fraction of unaffected cells (Fu)=1−Fa. The (Dx)₁ or (Dx)₂ can be readily calculated from the median-effect equation of Chou et al. (3−4)·Dx=Dm[Fa/(1−Fa)]^{1m}; where Dm is the median-effect dose that is obtained from the antilog of the X-intercept of the median-effect plot, X=log(D) versus Y=log [fa/(1−fa)] or Dm=10−(^{Y-intercept)/m}, and m is the slope of median-effect plot. Calcusyn software (Biosoft, Ferguson, Mo.) allows automated calculation of m, Dm, Dx, and CI values. From (Dm)₁, (Dx)₂, and D1+D2, isobolograms can be constructed based on the first equation.

Cytokine Profiling. Cell media were collected after treatment with 100 nM dasatinb or vehicle control and frozen at −80° C. until analysis. 100 μL of cell media was used in each well plate. A validated panel of 25 human cytokines/chemokines (Cytokine 25-plex antibody bead kit) was measured in duplicate using the Bioplex Protein Array Luminex 100 system (Biosource, Invitrogen Corp, Carlsbad, Calif.), according to manufacture's instructions. These included interleukin-1 beta (IL-1β), IL-1ra, IL-2, IL-2R, IL-4, IL-5, IL-6, IL-7, IL-8, IL-10, IL-12p40, IL-13, IL-15, IL-17, tumor necrosis factor-alpha (TNF-α), interferon-alpha (IFN-α), (IFN-γ), granulocyte-monocyte colony stimulating factor (GM-CSF), macrophage chemoattractant protein-1 (MCP-1), macrophage inflammatory protein 1α (MIP-1α), MIP-1β, inducible protein-10 (IP-10), MIG, Eotaxin, and RANTES. This is a multiplexed, particle-based, flow cytometric assay that utilizes anti-cytokine monoclonal antibodies linked to microspheres incorporating distinct proportions of two fluorescent dyes. For each cytokine calibration curves, eight standards ranged from 2.0 to 32,000 pg/mL.

Cell Cycle and Apoptosis Analysis. Subconfluent cells were treated with 100 nM dasatinib, 2.5 μM pyridine 6, or both for 6 h (apoptosis) or 24 and 48 h (cell cycle). Cells were also treated with nocadazole as a positive control for G2/M arrest. For cell cycle, cells were harvested, washed in phosphate-buffered saline (PBS), fixed in 1% paraformaldehyde, rewashed in PBS, and resuspended in 70% ethanol at −20° C. overnight. Cells were washed twice with PBS and stained with 20 μg/ml propidium iodide (PI). DNA content was analyzed on a cytofluorimeter by fluorescence-activated cell sorting analysis (FACScan; Becton Dickinson and Company, San Jose, Calif.) using ModFit software (Verity Software House, Turramurra, NSW, Australia). For apoptosis, treated cells were then harvested and stained with annexin V and PI and analyzed on a cytofluorimeter by FACScan using ModFit software.

### Results

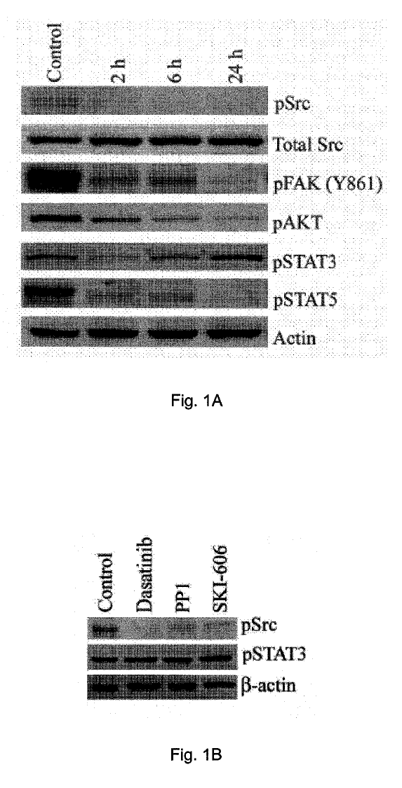

Src inhibition leads to initial STAT3 inhibition and later reactivation in multiple cancer cell types in culture. Fifteen human cancer cell lines were treated with 100 nM dasatinib for 0, 2 h, 6 h, and 24 h. Protein expression was measured by Western blot. In all cell lines c-Src was rapidly and durably inhibited. Additionally, several molecules downstream of Src (AKT, STAT5, and FAK) were also durably inhibited. In 14 of 15 cell lines tested [HNSCC (6/6), NSCLC (3/4), mesothelioma (3/3), and squamous skin carcinoma (3/3)] STAT3 activation was intitally inhibited but levels of pSTAT3 (Y105) returned to or above baseline by 24 h (FIG. 1A, and data not shown). One representative cell line was chosen for further investigation. In Tu167 cells (HNSCC cell line), treatment with 3 distinct SFK inhibitors all resulted in rapid (within 15 min, data not shown) and durable c-Src inhibition, but a re-activation of STAT3 (Y105) by 24 h. See FIG. 1B.

Reactivation of STAT3 is not mediated by activation of the EGFR pathway. STAT3 can be activated by growth factor or cytokine receptors coupled to the Src or JAK families of kinases. Yu, H. and Jove, R., Nat Rev Cancer, 4: 97-105, 2004. Dasatinib does not have any known direct stimulatory effect on growth factor or cytokine receptors Lombardo, L. J., Lee, F. Y., Chen, P., Norris, D., Barrish, J. C., Behnia, K., Castaneda, S., Cornelius, L. A., Das, J., Doweyko, A. M., Fairchild, C., Hunt, J. T., Inigo, I., Johnston, K., Kamath, A., Kan, D., Klei, H., Marathe, P., Pang, S., Peterson, R., Pitt, S., Schieven, G. L., Schmidt, R. J., Tokarski, J., Wen, M. L., Wityak, J., and Borzilleri, R. M., Discovery of N-(2-chloro-6-methyl-phenyl)-2-(6-(4-(2-hydroxyethyl)-piperazin-1-yl)-2-methylpyrimidin-4-ylamino)thiazole-5-carboxamide (BMS-354825), a Dual Src/Abl Kinase Inhibitor With Potent Antitumor Activity In Preclinical Assays, J Med Chem, 47: 6658-6661, 2004. We examined the effects of dasatinib on EGFR because this is a key growth factor pathway in several epithelial tumors and because of the extensive research that demonstrates that EGFR activation leads to STAT3 activation in HNSCC. Song, J. I. and Grandis, J. R., STAT Signaling in Head and Neck Cancer, Oncogene, 19: 2489-2495, 2000. Dasatinib treatment for 15 min with or without EGF did not affect EGFR activation in intact cells (FIG. 2A) which demonstrates that dasatinib does not directly affect EGFR in intact cells and confirms the in vitro kinase assay data. We hypothesized that SFK inhibition might lead to the indirect stimulation of EGFR and subsequent STAT3 reactivation based on the observation that in HNSCC cells treated with dasatinib, MAPK was transiently activated. Johnson, F. M., Saigal, B., Talpaz, M., and Donato, N.J., Clin Cancer Res, 11: 6924-6932, 2005. Tu167 cells were treated with an inhibitor of EGFR (erlotinib) which did not affect the STAT3 reactivation by dasatinib (FIG. 2B). Additionally, treatment of Tu167 cells with EGF only led to a slight increase in STAT3 activation. In contrast, MAPK was markedly activated by EGF. This suggests that STAT3 is not significantly affected by EGFR in these cells. In order to determine if MAPK activation lead to STAT3 activation in cells treated with dasatinib, cells were treated with an inhibitor of MAPK (PD98059) with no effect on STAT3 reactivation (data not shown). We also examined the effect of COX-2 inhibition because COX-2 can activate STAT3, but found no effect of COX-2 inhibitors on STAT3 baseline activation or re-activation at 24 h in these cells (data not shown). Dalwadi, H., Krysan, K., Heuze-Vourc'h, N., Dohadwala, M., Elashoff, D., Sharma, S., Cacalano, N., Lichtenstein, A., and Dubinett, S, Cyclooxygenase-2-Dependent Activation Of Signal Transducer And Activator Of Transcription 3 By Interleukin-6 In Non-Small Cell Lung Cancer, Clin Cancer Res, 11: 7674-7682, 2005. Stimulation of cells with insulin-like growth factor did not lead to significant activation of STAT3 nor did dasatinib affect IGF1R activation.

Reactivation of STAT3 is not mediated by cytokine release. In order to examine the effect of SFK inhibition on cytokine production, we examined the effect of 100 nM dasatinib on the production of 25 different cytokines in both serum-free and complete medium after 6 and 24 h of treatment. The results were similar in all treatment groups. The majority of cytokines and growth factors were undetectable [interleukin (IL)-2, IL-4, IL-5, IL-7, IL-13, IL-17, interferon-gamma, granulocyte-monocyte colony stimulating factor, macrophage inflammatory protein 1 alpha, macrophage inflammatory protein 1 beta, eotaxin, macrophage chemoattractant protein-1] or unaffected [IL-1beta, IL-12p40, IL-15, tumor necrosis factor (TNF)-alpha, interferon-alpha, inducible protein-10, MIG, RANTES, IL-10]. Two cytokines (IL-6, IL-8) were decreased by treatment with dasatinib (Table 1). No cytokine or growth factor assayed was significantly increased by dasatinib treatment. In addition, we transferred conditioned medium from Tu167 cells treated with dasatinib for 24 h to fresh cells and did not observe any STAT3 activation (data not shown). In toto, these data suggest that the reactivation of STAT3 is not mediated by a soluble factor, although an autocrine effect mediated by a secreted but unstable or cell-bound factor cannot be excluded.

In this study, we demonstrated that SFK inhibition leads to initial STAT3 inhibition but a reactivation of STAT3 at later time points in 14 of 15 cell lines tested including HNSCC, NSCLC, mesothelioma, and squamous carcinoma of the skin. STAT3 is reported to be activated by growth factor and cytokine receptors. We initially focused on the EGFR pathway because it is known to activate STAT3 in HNSCC and NSCLC and because of the transient activation of MAPK previously demonstrated in these cell lines. However, the mechanism of STAT3 reactivation does not involve the activation of EGFR or MAPK.

Given the intimate relationship between SFKs and STAT3 in HNSCC, the lack of sustained STAT3 inhibition with dasatinib treatment was surprising. The mechanism for STAT3 reactivation has not been fully elucidated. This may be a compensatory pathway activated by the cells to promote survival in the face of sustained SFK inhibition. The reactivation of STAT3 may be due to the effects of dasatinib on other targets. Although this would not be predicted by dasatinib's known targets, unpredicted molecular and biological effects do occur with other selective kinase inhibitors. For example, imatinib treatment can lead to MAPK activation in chronic myelogenous leukemia (CML) cells and to the release of HB-EGF and the subsequent activation of EGFR and MAPK in HNSCC cells. Yu, C., Krystal, G., Varticovksi, L., McKinstry, R., Rahmani, M., Dent, P., and Grant, S., Pharmacologic Mitogen-Activated Protein/Extracellular Signal-Regulated Kinase Kinase/Mitogen-Activated Protein Kinase Inhibitors Interact Synergistically With STI571 To Induce Apoptosis In Bcr/Abl-Expressing Human Leukemia Cells, Cancer Res, 62: 188-199, 2002; Johnson, F. M., Saigal, B., and Donato, N.J., Induction Of Heparin-Binding EGF-Like Growth Factor And Activation Of EGF Receptor In Imatinib Mesylate-Treated Squamous Carcinoma Cells, J Cell Physiol, 205: 218-227, 2005. Imatinib also reverses multi-drug resistance of CML cells by an unknown mechanism that requires prolonged exposure. Yeheskely-Hayon, D., Regev, R., Eytan, G. D., and Dann, E. J., The Tyrosine Kinase Inhibitors Imatinib And AG957 Reverse Multidrug Resistance In A Chronic Myelogenous Leukemia Cell Line, Leuk Res, 29: 793-802, 2005. The re-activation of STAT3 after treatment with distinct SFK inhibitor suggests that this is a target-specific effect; however none of these inhibitors is completely specific for SFKs.

Another surprising finding in this study was that EGFR activation or inhibition did not significantly affect STAT3 or c-Src in HNSCC cells. EGFR stimulation and inhibition did lead to expected MAPK (ERK1/2) activation and inhibition respectively. EGFR activation is linked to c-Src and STAT3 activation in other HNSCC cell lines and in patient tissues. STAT3 activation, demonstrated by increased dimer formation (STAT3:STAT3 and STAT3:STAT1) and increased phosphorylation, is common in HNSCC tissue specimens. Abrogation of either EGFR or TGF-alpha led to decreased STAT3 activation in HNSCC cell lines in vitro and in vivo. Hambek, M., Baghi, M., Strebhardt, K., May, A., Adunka, O., Gstottner, W., and Knecht, R., STAT 3 Activation In Head And Neck Squamous Cell Carcinomas Is Controlled By The EGFR, Anticancer Res, 24: 3881-3886, 2004; Song, J. I. and Grandis, J. R., Oncogene, 19: 2489-2495, 2000. However, c-Src and STAT3 activation are not always dependent on EGFR. In a panel of NSCLC cell lines that included those with both mutant or with EGFR, the effect of SFK inhibition (dasatinib) on STAT3 activation was modest to absent. Song, L., Morris, M., Bagui, T., Lee, F. Y., Jove, R., and Haura, E. B., Dasatinib (BMS-354825) Selectively Induces Apoptosis In Lung Cancer Cells Dependent On Epidermal Growth Factor Receptor Signaling For Survival, Cancer Res, 66: 5542-5548, 2006; Alvarez, J. V., Greulich, H., Sellers, W. R., Meyerson, M., and Frank, D. A., Signal Transducer And Activator Of Transcription 3 Is Required For The Oncogenic Effects Of Non-Small-Cell Lung Cancer-Associated Mutations Of The Epidermal Growth Factor Receptor, Cancer Res, 66: 3162-3168, 2006.

We claim:

1. A method of identifying cancer or an associated disorder comprising identifying and quantifying STAT3 occurring in a biological sample taken from a subject after administering a SFK inhibitor to said subject.

2. A method of detecting the reactivation of STAT3 in a subject comprising identifying STAT3 occurring in a biological sample taken from said subject after administering to said subject a SFK inhibitor.

3. A kit for use in determining treatment strategy for an individual with STAT-associated disorder, comprising
a means for determining whether a biological sample obtained from said individual has STAT reactivation; and optionally
instructions for use and interpretation of the kit results.
